(19) United States
(12) Patent Application Publication
(10) Pub. No.: US 2022/0415518 A1
(43) Pub. Date: Dec. 29, 2022

(54) DIGESTIVE SYSTEM SIMULATION AND PACING

(71) Applicant: Vektor Medical, Inc., Carlsbad, CA (US)

(72) Inventors: Robert Joseph Krummen, Bellevue, WA (US); Paul Jerome Krummen, Littleton, CO (US); Allen Kamrava, Beverly Hills, CA (US)

(21) Appl. No.: 17/809,521

(22) Filed: Jun. 28, 2022

Related U.S. Application Data
(60) Provisional application No. 63/216,333, filed on Jun. 29, 2021.

(51) Int. Cl. G16H 50/50; G06F 30/27; G16H 10/60

(52) U.S. Cl. CPC G16H 50/50; G06F 30/27; G16H 10/60

(57) ABSTRACT

Methods and systems for analyzing and treating digestive disorders (including gastrointestinal disorders) are provided. A system provides various technologies (e.g., machine learning and simulations) to support such analysis and treatment of digestive disorders. The system may employ computational modeling of the digestive system based on anatomical characteristics and electrical characteristics of the digestive system to simulate motion and electrical activity. The system generates representations of the electrical activity such as an electrogastrogram and generates mappings of the representations to characteristic values of the simulations. The system may train a machine learning model based on the mappings. When treating a patient, the mappings and/or the machine learning model may be employed to identify a patient characteristic based on a patient representation of electrical activity collected from the patient.

### CROSS-REFERENCE TO RELATED APPLICATION(S)

This application claims priority to the provisional application with Ser. No. 63/216,333 titled “Gastrointestinal Simulation and Pacing,” filed Jun. 29, 2021. The entire contents of the above noted provisional application are incorporated by reference as part of the disclosure of this document.

### BACKGROUND

Digestive disorders such as irritable bowel syndrome (IBS), diarrhea, and constipation have typically been treated by home remedies, diet modification, over-the-counter drugs, prescription drugs, and surgery. Although there has been success with such treatments for some patients, others remain less fortunate.

Some attempts have been made to use electrical stimulations of the gastrointestinal tract to treat these digestive disorders. One example is a gastric pacemaker that provides electrical stimulations at different points along the digestive system. One such gastric pacemaker is described in U.S. Pat. No. 5,690,691, entitled “Gastro-Intestinal Pacemaker having Phased Multi-Point Stimulation,” and issued on Nov. 25, 1997.

Another gastric pacemaker is described in “Colonic Pacing in the Treatment of Patients with Irritable Bowel Syndrome: Technique and Results,” by Shafik, A., et. al., Front Biosci, January 1, vol. 8, iss. 2, pp 1-5, 2003. Shafik describes that some patients with IBS had irregular slow wave rhythm and abnormal wave variables. Shafik describes implanting pacemakers in patients who could then control pacing of the colon. In some of the patients, when pacing stopped after six months of daily pacing, the improvement in the symptoms continued even without any pacing.

Yet another pacemaker, introduced by Medtronic, is a bowel control therapy system involving the implementation of a neurostimulator and a lead. The Medtronic system was originally designed for urinary and fecal incontinence. Although the system's neurostimulator and lead target only the most distal gastrointestinal nerves, current investigations are evaluating its efficacy in treating IBS and even potentially in inflammatory bowel disease. Once implanted, the bowel control therapy system stimulates the sacral nerve to target neural communication between the brain and bowel. The bowel control therapy system targets communication problems associated with the nerves that do not communicate correctly with the brain.

### BRIEF DESCRIPTION OF DRAWINGS

FIG. 1 is a block diagram that illustrates components of a digestive system analysis and treatment (DSAT) system in some embodiments.

FIG. 2 is a flow diagram that illustrates processing of a generate characteristics mapping component in some embodiments.

FIG. 3 is a flow diagram that illustrates a process of collecting and processing mesh readings in some embodiments.

FIG. 4 is a flow diagram that illustrates the processing of a generate ML model component in some embodiments.

### DETAILED DESCRIPTION

Methods and systems for analyzing and treating digestive disorders (including gastrointestinal disorders) are provided. A digestive system analysis and treatment (DSAT) system provides various technologies to support such analysis and treatment of digestive disorders. The DSAT system may employ computational modeling of the digestive system based on anatomical characteristics and electrical characteristics of the digestive system to simulate motion (e.g., of slow waves and peristalsis) and electrical activity (e.g., myoelectrical activity). The characteristics include the gastrointestinal migrating motor complex (MMC), action potentials, propagation velocity of slow waves, slow-wave threshold relating to activation of calcium channels, locations of abnormal electrical activity, neural signals, ablation locations, gastric wall thickness, colon shape and size, pacing location, pacing speed, patterns of electrical signal applied to the inner lining of the digestive system, disease state, location and characteristics of disease-causing tissue, structural remodeling, and so on.

The DSAT system runs simulations assuming different sets (or combinations) of characteristic values of characteristics (e.g., different pacing locations and different action potentials) to generate a characteristics mapping library that maps simulated data of each simulation to the set of characteristic values used in that simulation. The DSAT system models the digestive system using a three-dimensional (3D) mesh with the vertices (e.g., 50,000 vertices) representing locations within the digestive system. When modeling a colon, the 3D mesh may represent the ascending colon, transverse colon, descending colon, sigmoid colon, the rectosigmoid junction, the rectum, the anorectal junction, the anus, attributes of the enteric nervous system, rectoanal inhibitory reflex (RAIR), and so on. Each simulation may simulate activity at intervals, for example, of one second for simulation duration of 15 minutes. Such a 15-minute simulation at one-second intervals would generate 900 sets of mesh values for the 3D mesh. The values may include electrical activity, motion, and so on. After running a simulation, the DSAT system generates the simulated data that may include a simulated digestive electrogram (EDG) from the 900 sets of mesh values. An EDG may be an electrogastrogram (EGG), an electromyograph (EMG), an electrogastroenterogram, and so on, or their vector representations. Techniques for simulating electrical activity of the digestive system are described in various publications (e.g., Kawano M, Emoto T. Polygonally Meshed Dipole Model Simulation of the Electrical Field Produced by the Stomach and Intestines. Comput Math Methods Med. 2020 Oct. 21; 2020:2971358. doi: 10.1155/2020/2971358. PMID: 33178331; PMCID: PMC7607902.)

During a clinical study, a patient EDG is collected from a patient. To collect an EDG for a stomach, electrodes are placed (e.g., cutaneously) at various locations near the stomach. An EDG for a colon may be collected based on electrodes placed (e.g., transcutaneously or transmurally) at various locations relative to the colon. The DSAT system then compares the patient EDG to the simulated EDGs in the characteristics mapping library to find a matching (e.g., similar) simulated EDG. The DSAT system then outputs one or more characteristic values associated with the matching EDG to assist a physician in formulating an accurate assessment and plan for the patient such as performing an ablation or implanting a colon pacemaker.

In some embodiments, the characteristics mapping library may include mappings based on clinical data of patients. The mappings may map patient EDGs collected from patients to known characteristics of the patient digestive systems. These clinical mappings may be used to augment simulated mappings derived from the simulations to provide a larger collection of mappings. The DSAT system may use mappings that include only simulated mappings, only clinical mappings, or a combination of simulated and clinical mappings.

The DSAT system may also support monitoring electrical activity of the digestive system by collecting electrode readings based on placement of electrodes at various locations within the colon. For example, a catheter that is attached to a flexible and expandable tubular mesh (3D mesh) having an arrangement of electrodes may be inserted into the colon in a manner similar to insertion of a fiber-optic catheter during a colonoscopy. The catheter with the tubular electrode mesh may also include a fiber-optic component to guide placement of the tubular mesh. When the tubular mesh is placed at a desired location, the tubular mesh is expanded (e.g., by pulling a cable along the inside of the catheter) so that the electrodes contact the inner lining of the digestive system (e.g., the intestinal mucosa). The electrodes receive electrical signals generated based on electrical activity of the colon. From the electrical signals, mesh readings that represent electrical activity of the digestive system, such as electrical activity of the colon that causes motion of the colon, are collected. An EDG may be collected via electrode placed cutaneously on a patient while the mesh readings are collected. A clinical mesh mapping of the mesh readings to the collected EDG may be stored in a mesh mapping library. The DSAT system may generate simulated mesh readings for each simulation based on the simulated electrical activity represented by the 3D mesh. The mesh mapping library may include simulated mesh mappings, clinical mesh mappings (collected from patients), or both.

In some embodiments, the DSAT system provides information to help locate the position of a catheter within a colon based on the pacing locations of various simulations representing locations of electrical activations, such as locations along the mucosa of the colon. The DSAT system may run simulations with sets of characteristics that include a pacing location. The DSAT system generates a simulated EDG from each simulation that is stored in the characteristic mapping library. The characteristics mapping library may also include mappings based on clinical data with known pacing locations.

During a procedure such as an ablation or placement of a pacemaker, a catheter with an electrode near its tip may be used to track the location of the catheter. A medical provider directs movement of the catheter, directs the sending of electrical signals to the electrode while the electrode is in contact with (or in a vicinity of) the mucosa to stimulate electrical activity, and directs collecting of a patient EDG representing the simulated electrical activity. The DSAT system then identifies from the characteristics mapping library an EDG (simulated or clinical) that matches the patient EDG. The DSAT system outputs the location of the electrical activation associated with that matching EGG to inform a gastroenterologist of a possible location of the tip of the catheter. The gastroenterologist may use the location information to guide the catheter to a target location for treatment. The location information may be displayed on a graphic (e.g., a 3D image) of the digestive system. If a target path to the target location is provided in advance, that target path may be displayed on the graphic to assist so that a deviation from the target path can be visualized.

Once a potential target location for a treatment is identified, pacing may be employed to determine whether that potential target location is an appropriate target for the treatment (e.g., ablation or placement of a gastric pacemaker) based on, for example, review of a patient EDG collected during the pacing. The catheter may be moved to different potential target locations to identify the most appropriate location for the treatment. The potential target location of a pacing may be identified in an analogous manner as described above when guiding a catheter to a potential target location. A potential target location may be selected as the actual target location based on a patient EDG collected during the pacing at that potential target location and a prior patient EDG collected during an episode of a digestive disorder.

In some embodiments, the DSAT system provides information to help guide a therapy such as therapy that is an external body surface radiotherapy (ESBR, also known as stereotactic ablative radiotherapy) directed at specific organs such as the stomach, small intestine, large intestine, colon, and so forth. The DSAT system may be employed to predict the potential efficacy of a therapy (e.g., ablation) on the workings of the digestive system to assess treatment outcomes prior to performing the therapy. For example, the DSAT system may run a therapy simulation based on a set of characteristic values that is mapped to a simulated EDG that is similar to the patient EDG. Before running the therapy simulation, the set of characteristic values may be modified to represent the effect of the therapy and may also be modified to reflect some of the characteristics of the patient such as prior ablation locations, anatomical characteristics, and so on. For example, the target location of a therapy that is an ablation or pacing of a pacemaker may be added to the set of characteristics. The values of the 3D mesh used in the therapy simulation may be initialized to the values of the 3D mesh of the simulation from which the similar simulated EDG was generated. Thus, the therapy simulation is effectively a continuation of that similar simulation. After running the therapy simulation, the DSAT system may generate a therapy EDG based on the therapy simulation and output the therapy representation so that an assessment may be made of the efficacy of the therapy.

Similarity of EDGs (or mesh readings) may be based on an organ-specific scoring model, a similarity score, generated using various similarity measures such as a Pearson correlation or cosine similarity. If a similarity score satisfies a similarity criterion, the EDGs are considered to be similar. A similarity criterion may be a similarity score that exceeds a threshold similarity score, is the highest similarity score generated, and so on. The similarity criterion may be based on the intended application of the DSAT system, accuracy of the equipment that collects patient EDGs, analysis of clinical therapies that are successful, and so on.

In some cases, a patient may be under anesthesia during, for example, a colonic procedure. In such a case, there may be no or limited electrical activity within the colon. The DSAT system may be employed to stimulate electrical activity. For example, a catheter with an electrode at its tip may be inserted into the colon to activate the MMC electrical signals at a target location. A tubular electrode mesh that is similar to that used to monitor electrical activity may be used instead to stimulate electrical activity. For example, once the mesh is in position, the DSAT system may send electrical signals to the electrodes in a desired pattern (or more generally designated pattern) such as sending electrical signals in sequence from the proximal end of the ascending colon to the distal end of the ascending colon. The mesh may also be used to inform placement of a colon pacemaker. For example, an electrode can be paced, and an EDG can be collected. A gastroenterologist can assess the effectiveness of the pacing at that location based on review of the EDG as a possible location for an electrode of a pacemaker. The EDG may be generated, for example, based on electrodes placed cutaneously or electrodes of the expandable electrode mesh other than electrode(s) that are currently being used for pacing.

In some embodiments, the DSAT system employs a machine learning (ML) model to assist in the identification of a characteristic of the digestive system of a patient, referred to as an EDG to characteristic ML model. The DSAT system may train the ML model to input a patient EDG (and possibly characteristics of the patient such as prior ablation location and ablation pattern) and output a characteristic associated with that EDG. The characteristic may be a source location of digestive disorders (e.g., abnormal electrical activity), a type of digestive disorder, a treatment that was successful (or unsuccessful), patient demographics (e.g., age and sex), and so on. The training data may include data derived from the characteristics mapping library. The ML model (and those described below) may be trained using supervised or unsupervised training and may generate values of a discrete domain (e.g., classification), probabilities, and/or values of a continuous domain (e.g., regression value). When supervised training is used for any of the described ML models, the training data includes a feature vector with features that may include an EDG, data derived from the EDG (e.g., using principal components analysis, a latent feature vector, or an autoencoder), characteristic values of characteristics associated with an EDG, patient demographics, and so on and a label indicating the characteristic value (e.g., discrete value or continuous value) of a characteristic of interest. When supervised training is used, the ML model may be a convolution neural network, a support vector machine, recurrent neural network, and so on. When unsupervised training is used, the training data includes similar feature vectors such as those described above but without labels. Also, the ML model may be, for example, k-means clustering. The training data may be based on simulations and/or clinical data collected from patients such as the data of the characteristics mapping library.

The DSAT system may employ a ML model to map EDGs to mesh readings representing electrical activity of the digestive system, referred to as an EDG to mesh reading ML model. The mesh readings may be based on electrical signals collected from patients (e.g., invasively as described above) or derived from simulations. The training data for the ML model may include data derived from the mesh mapping library. The ML model may be trained using supervised or unsupervised training as described above. The mesh readings are associated with EDGs and with sets of characteristic values (used in generating simulated mesh readings or of a patient from whom the mesh readings were collected) such as those described above. The training data may include feature vectors that includes EDGs represented as a feature(s) labeled with associated mesh readings. After being trained, the ML model may be employed to input an EDG and output mesh readings. The EDG represents the EDG that would be collected while the mesh readings were collected. Thus, the mesh readings may help inform a treatment without having to invasively collect mesh readings.

The DSAT system may employ a ML model to map mesh readings to disease characteristics such as type of disease of the digestive system, referred to as mesh readings to disease ML model. The mesh readings may be based on electrical signals collected from patients (e.g., invasively as described above) or derived from simulations. The training data for the ML model may include data derived from the mesh mapping library. The ML model may be trained using supervised or unsupervised training as described above. The mesh readings are associated with disease characteristics such as type of disease, state of the disease, and so on (used in generating simulated mesh readings or of a patient from whom the mesh readings were collected) such as those described above. The training data may include feature vectors that include mesh readings represented as a feature(s) labeled with an associated disease characteristic. After being trained, the ML model may be employed to input mesh readings collected from a patient and output a disease characteristic. The output disease characteristic may represent a disease associated with the mesh readings and may help inform a treatment without having to invasively collect mesh readings. The ML models described above may be used in serial or in parallel such as the output of the EDG to mesh reading ML model being input to the mesh readings to disease ML model

The DSAT system provides techniques (as described herein) that allow a medical provider to develop a treatment plan in real time in a clinical setting during a procedure when a patient is being evaluated and treated. The mapping libraries and/or the ML models may be employed to provide information that may inform the treatment based on EDGs, mesh readings, or other data collected from the patient during that procedure. Because millions of simulations can be run using different sets of characteristic value, one or more simulations is highly likely to be a reasonably accurate representation of any patient. The simulations may run prior to treating any patient and may be used to inform treatment of any number of patients. Thus, the cost of running the simulations is incurred only once. In addition, the time and cost associated with collecting data from a patient and running a patient-specific simulation is avoided. Moreover, because of the inherent inaccuracies in collecting patient data, for example, because manual steps are involved or because of noise or accuracy of instruments, the DSAT system can produce more accurate and reliable information to inform treatment. Although the DSAT system is described primarily in the context of human patients, the DSAT system may be used with non-human patients such as horses and dogs.

The computing systems (e.g., network nodes or collections of network nodes) on which the DSAT system and the other described systems may be implemented may include a central processing unit, input devices, output devices (e.g., display devices and speakers), storage devices (e.g., memory and disk drives), network interfaces, graphics processing units, communications links (e.g., ethernet, WiFi, cellular, and Bluetooth), global positioning system devices, and so on. The input devices may include keyboards, pointing devices, touch screens, gesture recognition devices (e.g., for air gestures), head and eye tracking devices, microphones for voice recognition, and so on. The computing systems may include high-performance computing systems, cloud-based computing systems, client computing systems that interact with cloud-based computing systems, desktop computers, laptops, tablets, e-readers, personal digital assistants, smartphones, gaming devices, servers, and so on. The computing systems may access computer-readable media that include computer-readable storage media and data transmission media. The computer-readable storage media are tangible storage means that do not include a transitory, propagating signal. Examples of computer-readable storage media include memory such as primary memory, cache memory, and secondary memory (e.g., DVD) and other storage. The computer-readable storage media may have recorded on them or may be encoded with computer-executable instructions or logic that implements the DSAT system and the other described systems. The data transmission media are used for transmitting data via transitory, propagating signals or carrier waves (e.g., electromagnetism) via a wired or wireless connection. The computing systems may include a secure cryptoprocessor as part of a central processing unit for generating and securely storing keys and for encrypting and decrypting data using the keys.

The DSAT system and the other described systems may be described in the general context of computer-executable instructions, such as program modules and components, executed by one or more computers, processors, or other devices. Program modules or components include routines, programs, objects, data structures, and so on that perform tasks or implement data types of the DSAT system and the other described systems. Typically, the functionality of the program modules may be combined or distributed as desired in various examples. Aspects of the DSAT system and the other described systems may be implemented in hardware using, for example, an application-specific integrated circuit (ASIC) or field programmable gate array (FPGA).

At least some of the computer-executable instructions of the DSAT system execute on a server of a cloud system that receives and sends data to a client system. The client system may include a browser for receiving and display web page (e.g., with a graphic of the digestive system) and/or may include some instructions of the DSAT system.

A machine learning model may be any of a variety or combination of supervised or unsupervised machine learning models including neural networks such as fully connected, convolutional, recurrent, autoencoder, or restricted Boltzmann machine, a support vector machine, a Bayesian classifier, k-means clustering, and so on. When the machine learning model is a deep neural network, the training results are a set of weights for the activation functions of the deep neural network. A support vector machine operates by finding a hypersurface in the space of inputs. The hypersurface attempts to split the positive examples (e.g., feature vectors or photographs) from the negative examples (e.g., feature vectors for graphics) by maximizing the distance between the nearest of the positive and negative examples to the hypersurface. This step allows for correct classification of data that is similar to but not identical to the training data. A machine learning model may generate values of discrete domain (e.g., classification), probabilities, and/or values of a continuous domain (e.g., regression value).

Various techniques can be used to train a support vector machine. For example, adaptive boosting is an iterative process that runs multiple tests on a collection of training data. Adaptive boosting transforms a weak learning algorithm (an algorithm that performs at a level only slightly better than chance) into a strong learning algorithm (an algorithm that displays a low error rate). The weak learning algorithm is run on different subsets of the training data. The algorithm concentrates increasingly on those examples in which its predecessors tended to show mistakes. The algorithm corrects the errors made by earlier weak learners. The algorithm is adaptive because it adjusts to the error rates of its predecessors. Adaptive boosting combines rough and moderately inaccurate rules of thumb to create a high-performance algorithm. Adaptive boosting combines the results of each separately run test into a single, very accurate classifier. Adaptive boosting may use weak classifiers that are single-split trees with only two leaf nodes.

A neural network model has three major components: architecture, cost function, and search algorithm. The architecture defines the functional form relating the inputs to the outputs (in terms of network topology, unit connectivity, and activation functions). The search in weight space for a set of weights that minimizes the objective function is the training process. In one embodiment, the classification system may use a radial basis function (RBF) network and a standard gradient descent as the search technique.

A convolutional neural network (CNN) has multiple layers such as a convolutional layer, a rectified linear unit (ReLU) layer, a pooling layer, a fully connected (FC) layer, and so on. Some more complex CNNs may have multiple convolutional layers, ReLU layers, pooling layers, and FC layers.

A convolutional layer may include multiple filters (also referred to as kernels or activation functions). A filter inputs a convolutional window, for example, of an image, applies weights to each pixel of the convolutional window, and outputs an activation value for that convolutional window. For example, if the static image is 256 by 256 pixels, the convolutional window may be 8 by 8 pixels. The filter may apply a different weight to each of the 64 pixels in a convolutional window to generate the activation value also referred to as a feature value. The convolutional layer may include, for each filter, a node (also referred to as a neuron) for each pixel of the image assuming a stride of one with appropriate padding. Each node outputs a feature value based on a set of weights for the filter that are learned during training.

The ReLU layer may have a node for each node of the convolutional layer that generates a feature value. The generated feature values form a ReLU feature map. The ReLU layer applies a filter to each feature value of a convolutional feature map to generate feature values for a ReLU feature map. For example, a filter such as max(0, activation value) may be used to ensure that the feature values of the ReLU feature map are not negative.

The pooling layer may be used to reduce the size of the ReLU feature map by downsampling the ReLU feature map to form a pooling feature map. The pooling layer includes a pooling function that inputs a group of feature values of the ReLU feature map and outputs a feature value.

The FC layer includes some number of nodes that are each connected to every feature value of the pooling feature maps.

A generative adversarial network (GAN) or an attribute (attGAN) may also be used. An attGAN employs a GAN to train the generator model. (See, Zhenliang He, Wangmeng Zuo, Meina Kan, Shiguang Shan, and Xilin Chen, “AttGAN: Facial Attribute Editing by Only Changing What You Want,” IEEE Transactions on Image Processing, 2019; and Ian Goodfellow, Jean Pouget-Abadie, Mehdi Mirza, Bing Xu, David Warde-Farley, Sherjil Ozair, Aaron Courville, and Yoshua Bengio, “Generative Adversarial Nets,” Advances in Neural Information Processing Systems, pp. 2672-2680, 2014, which are hereby incorporated by reference.) An attribute GAN includes a generator and discriminator and an attribute GAN classifier and is trained using training data that includes input images of objects and input attribute values of each object. The generator includes a generator encoder and a generator decoder. The generator encoder inputs an input image and is trained to generate a latent vector of latent variables representing the input image. The generator decoder inputs the latent vector for an input image and the input attribute values. The attribute GAN classifier inputs an image and generates a prediction of its attribute values. The attribute GAN is trained to generate a modified image that represents the input image modified based on the attribute values. The generator encoder and the generator decoder form the generator model.

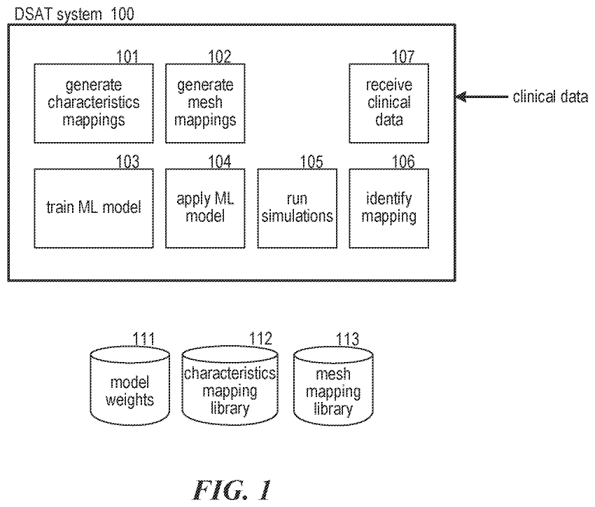

FIG. 1 is a block diagram that illustrates components of the DSAT system in some embodiments. The DSAT system 100 includes a generate characteristics mappings component 101, a generate mesh mappings component 102, a train machine learning model component 103, an apply machine learning model component 104, a run simulations component 105, an identify mappings component 106, and a receive clinical data component 107. The DSAT system also interfaces with model weights data store 111, a characteristics mapping library 112, and a mesh mapping library 113. The generate characteristics mappings component generates a characteristics mapping library that maps EDGs to corresponding characteristics. The generate mesh mappings component generates a mesh mapping library that maps EDGs to mesh readings. The train ML model component trains an ML model based on training data that includes the characteristics mapping library or the mesh mapping library. The train ML model component may store the data that is learned, such as weights of activation functions or clustering data in the model weights data store. The apply ML model component receives an input (features used in training) and applies a trained ML model to identify a characteristic (e.g., disease characteristic) or mesh readings associated with the input. The run simulations component run simulations of electrical activity based on sets of characteristic values and generates EDGs, mesh readings, and/or other representations of electrical activity based on the simulations. The identify mapping component receives characteristic values, EDGs, or mesh readings and employs the characteristics mapping library to identify a characteristic or employ the mesh mapping library to identify mesh readings given an EDG and/or characteristic values associated with the mesh readings. The receive clinical data component receives clinical data that may be stored in the characteristics mapping library or the mesh mapping library.

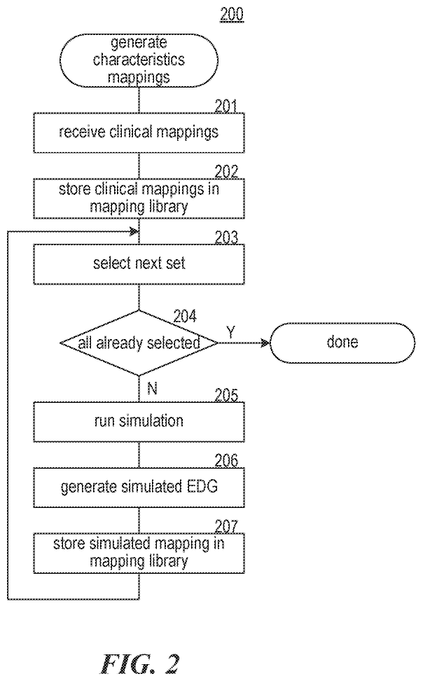

FIG. 2 is a flow diagram that illustrates processing of a generate characteristics mapping component 200 in some embodiments. The generate characteristics mapping component generates a characteristics mapping library that maps EDGs to characteristics based on clinical data and/or simulated data. In block 201, the component receives mappings of clinical data. In block 202, the component stores the clinical mappings in the characteristics mapping library. In block 203-207, the component runs simulations to generate simulated EDGs. In block 203, the component selects a next set of characteristic values. In decision block 204, if all the sets of characteristic values have already been selected, then the component completes, else the component continues at block 205. In block 205, the component runs a simulation based on the set of characteristic values. In block 206, the component generates a simulated EDG and/or simulated mesh readings based on the simulation. In block 207, the component stores a mapping of the simulated EDG or simulated mesh reading to a characteristic(s) of the set of characteristic values in the characteristics mapping library. The component then loops to block 203 to select the next set of characteristic values.

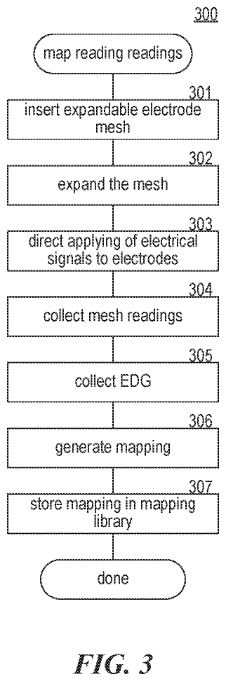

FIG. 3 is a flow diagram that illustrates a process of collecting and processing mesh readings in some embodiments. The process 300 involves activity of a person to control placement of an electrode mesh and a computer system to generate mappings. In block 301, a person inserts an expandable electrode mesh into the digestive system of a patient. In block 302, the person controls the expanding of the mesh so that the electrodes contact the inner lining of the digestive system. In some embodiments, in block 303, the person may direct the applying of electrical signals to the electrodes to help control electrical activity of the digestive system. In block 304, an EDG and mesh readings are collected. In block 306, mappings of the EDG to the mesh readings are generated. In block 307, the mappings are stored in the mesh mapping library.

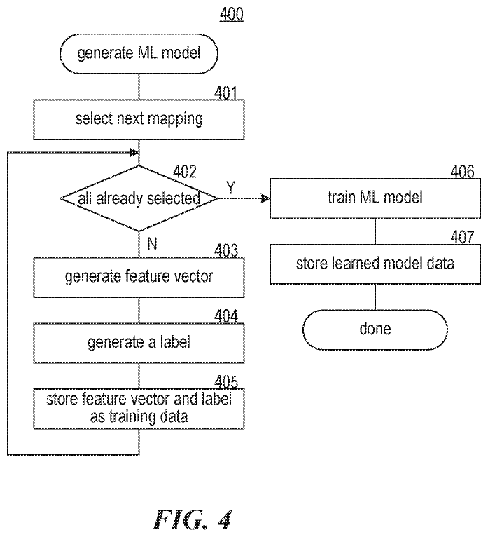

FIG. 4 is a flow diagram that illustrates the processing of a generate ML model component in some embodiments. The generate ML model component 400 trains a ML model based on the characteristics mapping library or the mesh mapping library or both. In block 401, the component selects the next mapping. In decision block 402, if all the mappings have already been selected, then the component continues at block 406, otherwise the component continues at block 403. In block 403, the component generates a feature vector based on the selected mapping. In block 404, the component generates a label for the feature vector (assuming supervised learning). In block 405, the component stores the feature vector and the label as the training data and then loops to block 401 to select the next mapping. In block 406, the component trains the ML model based on the training data. In block 407, the component stores the learned model data such as weights or clustering data in a model weight data store and then completes.

The following paragraphs describe various embodiments of aspects of the DSAT system. An implementation of the system may employ any combination of the embodiments. The processing described below may be performed by a computing system with a processor that executes computer-executable instructions stored on a computer-readable storage medium that implements the system.

In some aspects, the techniques described herein relate to a method performed by one or more computing systems for modeling electrical activity of a digestive system, the method including: running simulations to simulate electrical activity of the digestive system, each simulation based on a set of characteristic values of characteristics of the digestive system; for each of a plurality of simulations, generating a simulated digestive electrogram (EDG) representing electrical activity of the digestive system based on the simulated electrical activity of that simulation; and generating a characteristics mapping library that includes mappings of the simulated EDGs to one or more characteristic values of the set of characteristic values of the simulation from which the simulated EDGs were generated.

In some aspects, the techniques described herein relate to a method further including training a machine learning model to output a characteristic value representing a characteristic given an EDG, the machine learning model being trained using the mappings of the characteristics mapping library.

In some aspects, the techniques described herein relate to a method wherein the characteristic value that is output by the machine learning model is a value of a discrete domain.

In some aspects, the techniques described herein relate to a method wherein the characteristic value that is output by the machine learning model is a value of a continuous domain.

In some aspects, the techniques described herein relate to a method further including: receiving a patient EDG collected from a patient; inputting the patient EDG into a machine learning model to generate an output indicating a characteristic value for a characteristic of the patient, the machine learning model being trained based on mappings of the characteristics mapping library; and outputting an indication of the indicated characteristic value of the characteristic.

In some aspects, the techniques described herein relate to a method wherein the characteristics mapping library includes mappings of clinical EDGs collected from patients to one or more characteristic values representing characteristics of the patients.

In some aspects, the techniques described herein relate to a method wherein a characteristic is a source location of abnormal electrical activity.

In some aspects, the techniques described herein relate to a method further including: receiving a patient EDG collected from a patient; identifying a simulated EDG of the characteristics mapping library that is similar to the patient EDG based on satisfying a similarity criterion; and outputting an indication of a characteristic value of a characteristic that is mapped to the identified simulated EDG.

In some aspects, the techniques described herein relate to a method further including guiding a catheter within the digestive system by, for each of a plurality of pacing locations, receiving pacing EDGs while pacing at the pacing location, determining the pacing location based on a simulated EDG that is similar to a patient EDG based on satisfying a similarity criterion, and outputting the determined pacing location.

In some aspects, the techniques described herein relate to a method further including guiding a catheter within the digestive system by, for each of a plurality of pacing locations, receiving pacing EDGs while pacing at the pacing location, inputting the pacing EDG to a machine learning model that outputs a pacing location, and outputting the output pacing location.

In some aspects, the techniques described herein relate to a method further including displaying an indication of the determined pacing location on an image of a digestive system.

In some aspects, the techniques described herein relate to a method performed by one or more computing systems for generating a representation of electrical activity of a digestive system, the method including: directing insertion of an expandable electrode mesh into the digestive system of a patient, the expandable electrode mesh having a plurality of electrodes; after the expandable electrode mesh is expanded so that electrodes contact the inner lining of the digestive system, receiving mesh readings generated from electrical signals received via the electrodes; and receiving a digestive electrogram (EDG) collected while the electrical signals are received via the electrodes of the expandable electrode mesh.

In some aspects, the techniques described herein relate to a method wherein the expandable electrode mesh has a tubular shape prior to being expanded into a three-dimensional mesh.

In some aspects, the techniques described herein relate to a method wherein the expandable electrode mesh is expanded by pulling a cable that is inside a catheter to which the expandable electrode mesh is attached.

In some aspects, the techniques described herein relate to a method further including training a machine learning model with training data that includes EDGs labeled with mesh readings.

In some aspects, the techniques described herein relate to a method further including applying the machine learning model to a patient EDG collected from a patient wherein the machine learning model outputs an indication of mesh readings.

In some aspects, the techniques described herein relate to a method further including directing the applying of electrical signals to an electrode of the expandable electrode mesh to stimulate electrical activity of the digestive system while other electrodes receive electrical signals from which mesh readings are generated.

In some aspects, the techniques described herein relate to a method further including running simulations of electrical activity of a digestive system, generating simulated mesh readings and simulated EDGs based on the simulated electrical activity, and generating a mesh mapping library that includes mappings of the simulated EDGs to the simulated mesh readings.

In some aspects, the techniques described herein relate to a method for stimulating electrical activity of a patient digestive system of a patient, the method including: inserting an expandable electrode mesh into the patient digestive system, the expandable electrode mesh having electrodes; expanding the expandable electrode mesh so that the electrodes contact the inner lining of the patient digestive system; and directing electrical signals to be sent to the electrodes in a designated pattern to stimulate electrical activity of the patient digestive system.

In some aspects, the techniques described herein relate to a method wherein the patient is under anesthesia.

In some aspects, the techniques described herein relate to a method wherein the expandable electrode mesh has a tubular shape prior to being expanded into a three-dimensional mesh.

In some aspects, the techniques described herein relate to a method wherein the expandable electrode mesh is expanded by pulling a cable that is inside a catheter to which the expandable electrode mesh is attached.

In some aspects, the techniques described herein relate to a method further including analyzing a patient digestive electrogram (EDG) collected during the stimulated electrical activity.

In some aspects, the techniques described herein relate to a method wherein the analyzing includes reviewing patient characteristic values of characteristics retrieved from a mapping library that maps EDGs to characteristic values, the patient characteristic values being mapped to an EDG of the mapping library that is similar to the patient EDG.

In some aspects, the techniques described herein relate to a method wherein at least one of the electrodes receives a signal generated by the digestive system in response to the stimulated electrical activity.

In some aspects, the techniques described herein relate to one or more computing systems that model electrical activity of a digestive system, the one or more computing systems including: one or more computer-readable storage mediums that store computer-executable instructions for controlling the one or more computing systems to: run simulations to simulate electrical activity of the digestive system, each simulation based on a set of characteristic values of characteristics of the digestive system; for each of a plurality of simulations, generate a simulated representation of electrical activity of the digestive based on the simulated electrical activity of that simulation; and generate a characteristics mapping library that includes mappings of the simulated representations of electrical activity to one or more characteristic values of the set of characteristic values used in the simulation from which the simulated representation of electrical activity was generated; and one or more processors for controlling the one or more computing systems to execute the one or more computer-executable instructions.

In some aspects, the techniques described herein relate to one or more computing systems wherein the computer-executable instructions further include instructions to train a machine learning model to output a characteristic value representing a characteristic given a representation of electrical activity, the machine learning model being trained using the mappings of the characteristics mapping library.

In some aspects, the techniques described herein relate to one or more computing systems for identifying a patient characteristic of a patient digestive system of a patient, the one or more computing systems including: one or more computer-readable storage mediums that store computer-executable instructions for controlling the one or more computing systems to: access a characteristics mapping library that includes mappings of library representations of electrical activity of the digestive system to characteristic values of characteristics of the digestive system; receive a patient representation of electrical activity collected from the patient; identify a library representation of electrical activity that is similar to the patient representation of electrical activity based on a similarity criterion; and output an indication of a characteristic value to which the identified library representation of electrical activity is mapped; and one or more processors for controlling the one or more computing systems to execute the one or more computer-executable instructions.

In some aspects, the techniques described herein relate to one or more computing systems wherein at least one of the computing systems is a cloud-based computing system that executes the instructions.

In some aspects, the techniques described herein relate to one or more computing systems wherein the patient representation of electrical activity is received from a client computing system.

In some aspects, the techniques described herein relate to one or more computing systems wherein the characteristics mapping library includes mappings based on simulated electrical activity of the digestive system.

In some aspects, the techniques described herein relate to one or more computing systems wherein the characteristics mapping library includes mappings based on clinical representations of electrical activity collected from patients.

In some aspects, the techniques described herein relate to a method for guiding a catheter within the digestive system of a patient, the method including: inserting the catheter into the digestive system of the patient, the catheter having an electrode for stimulating electrical activity; and for each of a plurality of locations within the digestive system, placing the electrode in contact with the mucosa of the digestive system; directing the electrode to stimulate electrical activity of the digestive system; collecting a digestive electrogram (EDG) based on the stimulated electrical activity; receiving an indication of the location of the electrode, the location determined based on mappings of EDGs to locations; and directing movement of the electrode to another location.

In some aspects, the techniques described herein relate to a method wherein the catheter is guided to a target location.

In some aspects, the techniques described herein relate to a method wherein the EDG is input to a device that outputs the location of the electrode.

In some aspects, the techniques described herein relate to a method wherein the location is determined by a computing system that inputs the EDG to a machine learning (ML) model that outputs the location, the ML model trained with training data derived from the mappings.

In some aspects, the techniques described herein relate to a method wherein indication is displayed on a digestive system graphic at the location.

In some aspects, the techniques described herein relate to one or more computing systems for determining a location of an electrode with a digestive system of a patient, the one or more computing systems including: one or more computer-readable storage mediums that store computer-executable instructions for controlling the one or more computing systems to: receive a digestive electrogram (EDG) that was collected while an electrode within the digestive system of the patient stimulates electrical activity of the digestive system; determine the location of the electrode based on mappings of EDGs to location; and output an indication of the determined location. one or more processors for controlling the one or more computing systems to execute the one or more computer-executable instructions.

In some aspects, the techniques described herein relate to one or more computing systems wherein the location is determined by a computing system that inputs the EDG to a machine learning (ML) model that outputs the location, the ML model trained with training data derived from the mappings.

In some aspects, the techniques described herein relate to a method for stimulating electrical activity of a patient digestive system of a patient under anesthesia, the method including: inserting an expandable electrode mesh into the patient digestive system, the expandable electrode mesh having electrodes; expanding the expandable electrode mesh so that the electrodes contact the inner lining of the patient digestive system; directing electrical signals to be sent to the electrodes in a designated pattern to stimulate electrical activity of the patient digestive system; and analyzing a patient digestive electrogram (EDG) collected during the stimulated electrical activity.

Although the subject matter has been described in language specific to structural features and/or acts, it is to be understood that the subject matter defined in the appended claims is not necessarily limited to the specific features or acts described above. Rather, the specific features and acts described above are disclosed as example forms of implementing the claims.

1. A method performed by one or more computing systems for modeling electrical activity of a digestive system, the method comprising:
running simulations to simulate electrical activity of the digestive system, each simulation based on a set of characteristic values of characteristics of the digestive system;
for each of a plurality of simulations, generating a simulated digestive electrogram (EDG) representing electrical activity of the digestive system based on the simulated electrical activity of that simulation; and
generating a characteristics mapping library that includes mappings of the simulated EDGs to one or more characteristic values of the set of characteristic values of the simulation from which the simulated EDGs were generated.

2. The method of claim 1 further comprising training a machine learning model to output a characteristic value representing a characteristic given an EDG, the machine learning model being trained using the mappings of the characteristics mapping library.

3. The method of claim 2 wherein the characteristic value that is output by the machine learning model is a value of a discrete domain.

4. The method of claim 2 wherein the characteristic value that is output by the machine learning model is a value of a continuous domain.

5. The method of claim 1 further comprising:
receiving a patient EDG collected from a patient;
inputting the patient EDG into a machine learning model to generate an output indicating a characteristic value for a characteristic of the patient, the machine learning model being trained based on mappings of the characteristics mapping library; and
outputting an indication of the indicated characteristic value of the characteristic.

6. The method of claim 1 wherein the characteristics mapping library includes mappings of clinical EDGs collected from patients to one or more characteristic values representing characteristics of the patients.

7. The method of claim 1 wherein a characteristic is a source location of abnormal electrical activity.

8. The method of claim 1 further comprising:
receiving a patient EDG collected from a patient;
identifying a simulated EDG of the characteristics mapping library that is similar to the patient EDG based on satisfying a similarity criterion; and
outputting an indication of a characteristic value of a characteristic that is mapped to the identified simulated EDG.

9. The method of claim 1 further comprising guiding a catheter within the digestive system by, for each of a plurality of pacing locations, receiving pacing EDGs while pacing at the pacing location, determining the pacing location based on a simulated EDG that is similar to a patient EDG based on satisfying a similarity criterion, and outputting the determined pacing location.

10. The method of claim 1 further comprising guiding a catheter within the digestive system by, for each of a plurality of pacing locations, receiving pacing EDGs while pacing at the pacing location, inputting the pacing EDG to a machine learning model that outputs a pacing location, and outputting the output pacing location.

11. The method of claim 10 further comprising displaying an indication of the determined pacing location on an image of a digestive system.

12. A method performed by one or more computing systems for generating a representation of electrical activity of a digestive system, the method comprising:
directing insertion of an expandable electrode mesh into the digestive system of a patient, the expandable electrode mesh having a plurality of electrodes;
after the expandable electrode mesh is expanded so that electrodes contact the inner lining of the digestive system, receiving mesh readings generated from electrical signals received via the electrodes; and
receiving a digestive electrogram (EDG) collected while the electrical signals are received via the electrodes of the expandable electrode mesh.

13. The method of claim 12 wherein the expandable electrode mesh has a tubular shape prior to being expanded into a three-dimensional mesh.

14. The method of claim 12 wherein the expandable electrode mesh is expanded by pulling a cable that is inside a catheter to which the expandable electrode mesh is attached.

15. The method of claim 12 further comprising training a machine learning model with training data that includes EDGs labeled with mesh readings.

16. The method of claim 15 further comprising applying the machine learning model to a patient EDG collected from a patient wherein the machine learning model outputs an indication of mesh readings.

17. The method of claim 16 further comprising directing the applying of electrical signals to an electrode of the expandable electrode mesh to stimulate electrical activity of the digestive system while other electrodes receive electrical signals from which mesh readings are generated.

18. The method of claim 12 further comprising running simulations of electrical activity of a digestive system, generating simulated mesh readings and simulated EDGs based on the simulated electrical activity, and generating a mesh mapping library that includes mappings of the simulated EDGs to the simulated mesh readings.

19. A method for stimulating electrical activity of a patient digestive system of a patient, the method comprising:
inserting an expandable electrode mesh into the patient digestive system, the expandable electrode mesh having electrodes;
expanding the expandable electrode mesh so that the electrodes contact the inner lining of the patient digestive system; and
directing electrical signals to be sent to the electrodes in a designated pattern to stimulate electrical activity of the patient digestive system.

20. The method of claim 19 wherein the patient is under anesthesia.

21. The method of claim 19 wherein the expandable electrode mesh has a tubular shape prior to being expanded into a three-dimensional mesh.

22. The method of claim 19 wherein the expandable electrode mesh is expanded by pulling a cable that is inside a catheter to which the expandable electrode mesh is attached.

23. The method of claim 19 further comprising analyzing a patient digestive electrogram (EDG) collected during the stimulated electrical activity.

24. The method of claim 23 wherein the analyzing includes reviewing patient characteristic values of characteristics retrieved from a mapping library that maps EDGs to characteristic values, the patient characteristic values being mapped to an EDG of the mapping library that is similar to the patient EDG.

25. The method of claim 19 wherein at least one of the electrodes receives a signal generated by the digestive system in response to the stimulated electrical activity.

26. One or more computing systems that model electrical activity of a digestive system, the one or more computing systems comprising:
one or more computer-readable storage mediums that store computer-executable instructions for controlling the one or more computing systems to:
run simulations to simulate electrical activity of the digestive system, each simulation based on a set of characteristic values of characteristics of the digestive system;
for each of a plurality of simulations, generate a simulated representation of electrical activity of the digestive based on the simulated electrical activity of that simulation; and
generate a characteristics mapping library that includes mappings of the simulated representations of electrical activity to one or more characteristic values of the set of characteristic values used in the simulation from which the simulated representation of electrical activity was generated; and
one or more processors for controlling the one or more computing systems to execute the one or more computer-executable instructions.

27. The one or more computing systems of claim 26 wherein the computer-executable instructions further include instructions to train a machine learning model to output a characteristic value representing a characteristic given a representation of electrical activity, the machine learning model being trained using the mappings of the characteristics mapping library.

28. One or more computing systems for identifying a patient characteristic of a patient digestive system of a patient, the one or more computing systems comprising:
one or more computer-readable storage mediums that store computer-executable instructions for controlling the one or more computing systems to:
access a characteristics mapping library that includes mappings of library representations of electrical activity of the digestive system to characteristic values of characteristics of the digestive system;
receive a patient representation of electrical activity collected from the patient;
identify a library representation of electrical activity that is similar to the patient representation of electrical activity based on a similarity criterion; and
output an indication of a characteristic value to which the identified library representation of electrical activity is mapped; and
one or more processors for controlling the one or more computing systems to execute the one or more computer-executable instructions.

29. The one or more computing systems of claim 28 wherein at least one of the computing systems is a cloud-based computing system that executes the instructions.

30. The one or more computing systems of claim 29 wherein the patient representation of electrical activity is received from a client computing system.

31. The one or more computing systems of claim 28 wherein the characteristics mapping library includes mappings based on simulated electrical activity of the digestive system.

32. The one or more computing systems of claim 28 wherein the characteristics mapping library includes mappings based on clinical representations of electrical activity collected from patients.

33. A method for guiding a catheter within the digestive system of a patient, the method comprising:
inserting the catheter into the digestive system of the patient, the catheter having an electrode for stimulating electrical activity; and
for each of a plurality of locations within the digestive system,
placing the electrode in contact with the mucosa of the digestive system;
directing the electrode to stimulate electrical activity of the digestive system;
collecting a digestive electrogram (EDG) based on the stimulated electrical activity;
receiving an indication of the location of the electrode, the location determined based on mappings of EDGs to locations; and
directing movement of the electrode to another location.

34. The method of claim 33 wherein the catheter is guided to a target location.

35. The method of claim 33 wherein the EDG is input to a device that outputs the location of the electrode.

36. The method of claim 33 wherein the location is determined by a computing system that inputs the EDG to a machine learning (ML) model that outputs the location, the ML model trained with training data derived from the mappings.

37. The method of claim 33 wherein indication is displayed on a digestive system graphic at the location.

38. One or more computing systems for determining a location of an electrode with a digestive system of a patient, the one or more computing systems comprising:
one or more computer-readable storage mediums that store computer-executable instructions for controlling the one or more computing systems to:
receive a digestive electrogram (EDG) that was collected while an electrode within the digestive system of the patient stimulates electrical activity of the digestive system;
determine the location of the electrode based on mappings of EDGs to location; and
output an indication of the determined location.
one or more processors for controlling the one or more computing systems to execute the one or more computer-executable instructions.

39. The one or more computing systems of claim 38 wherein the location is determined by a computing system that inputs the EDG to a machine learning (ML) model that outputs the location, the ML model trained with training data derived from the mappings.

40. A method for stimulating electrical activity of a patient digestive system of a patient under anesthesia, the method comprising:
inserting an expandable electrode mesh into the patient digestive system, the expandable electrode mesh having electrodes;
expanding the expandable electrode mesh so that the electrodes contact the inner lining of the patient digestive system;
directing electrical signals to be sent to the electrodes in a designated pattern to stimulate electrical activity of the patient digestive system; and
analyzing a patient digestive electrogram (EDG) collected during the stimulated electrical activity.
